(19) United States
(12) Patent Application Publication
(10) Pub. No.: US 2005/0059819 A1
(43) Pub. Date: Mar. 17, 2005

(54) Cefdinir pyridine salt

(76) Inventors: Richard W. Duerst, Lake Bluff, IL (US); Devalina Law, Libertyville, IL (US); Xiaochun Lou, Long Grove, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/778,851

(22) Filed: Feb. 13, 2004

Related U.S. Application Data
(63) Continuation-in-part of application No. 10/661,148, filed on Sep. 12, 2003.

(51) Int. Cl. C07D 501/14

(52) U.S. Cl. 540/222

(57) ABSTRACT

The present invention relates to a novel pyridine salt of 7-[2-(2-aminothiazol-4-yl)-2-hydroxyiminoacetamide]-3-vinyl-3-cephem-4-carboxylic acid (syn isomer), methods for its preparation, and pharmaceutical compositions comprising the salt.

This application is a continuation-in-part of U.S. patent application Ser. No. 10/661,148, filed Sep. 12, 2003, which is hereby incorporated by reference.

### TECHNICAL FIELD

The present invention relates to a novel pyridine salt of 7-[2-(2-aminothiazol-4-yl)-2-hydroxyiminoacetamide]-3-vinyl-3-cephem-4-carboxylic acid (syn isomer), methods for its preparation, and pharmaceutical compositions comprising the novel crystalline salt.

### BACKGROUND OF THE INVENTION

The antimicrobial agent 7-[2-(2-aminothiazol-4-yl)-2-hydroxyiminoacetamido]-3-vinyl-3-cephem-4-carboxylic acid (syn isomer) (hereinafter referred to as “Cefdinir”) is a semi-synthetic oral antibiotic in the cephalosporin family. Cefdinir is sold in the United States as Omnicef® in capsule and oral suspension forms. Omnicef® is active against a wide spectrum of bacteria, including Staphylococcus aureus, Streptococcus pneumoniae, Streptococcus pogenes, Hemophilus influenzae, Moraxella catarrhalis, E. coli, Klebsiella, and Proteus mirabilis. The preparation of Cefdinir was first disclosed in U.S. Pat. No. 4,559,334, issued Dec. 17, 1985, while the preparation of the commercially available form of Cefdinir (Crystal A or Form I) was first disclosed in U.S. Pat. No. 4,935,507, issued Jun. 19, 1990, both of which are hereby incorporated by reference in their entirety.

The present invention provides a novel pyridine salt of Cefdinir as well as pharmaceutical compositions and uses thereof. Pharmaceutical compositions comprising cefdinir and its salts are useful in treating bacterial infections such as Streptococcus pneumoniae and Hemophilus influenzae.

### BRIEF DESCRIPTION OF THE FIGURE

FIG. 1 is a representative powder X-ray diffraction pattern of the Form I of Cefdinir.

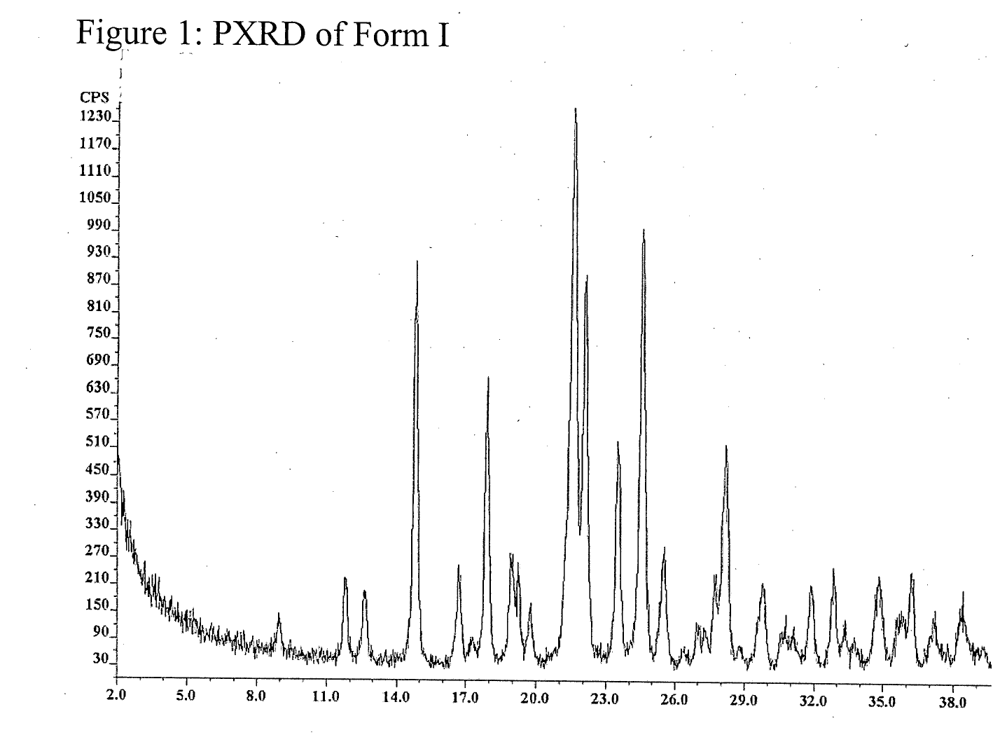

FIG. 2 is a representative powder X-ray diffraction pattern of the pyridine salt of Cefdinir.

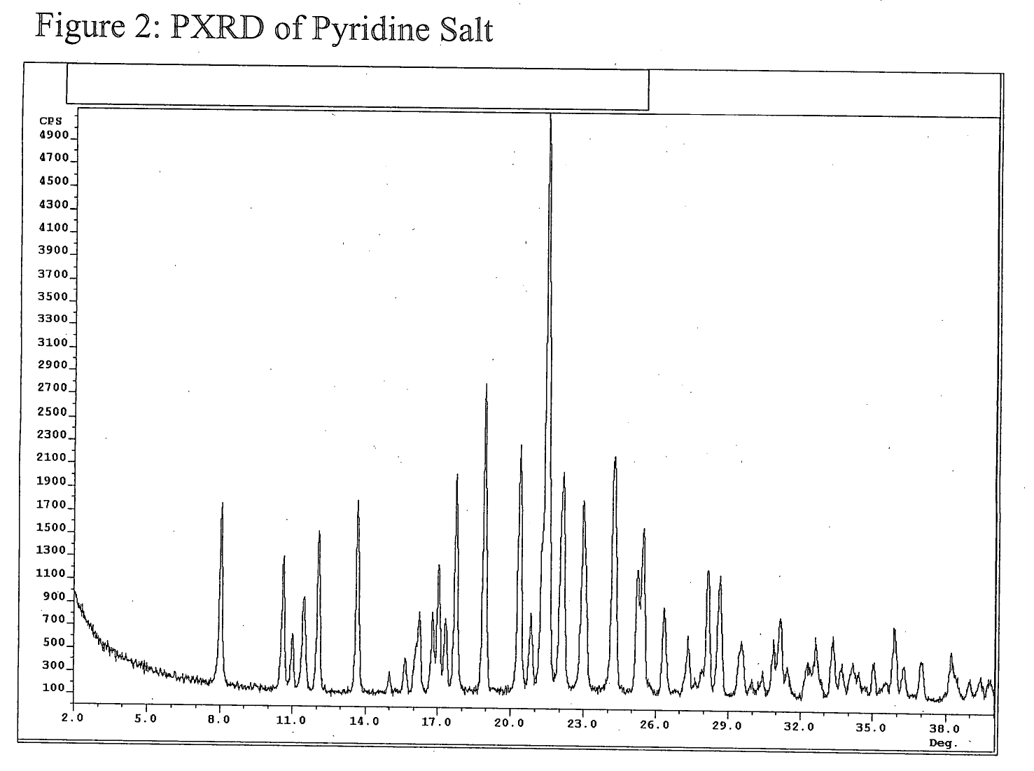

FIG. 3 is the infrared spectrum of the Form I of Cefdinir.

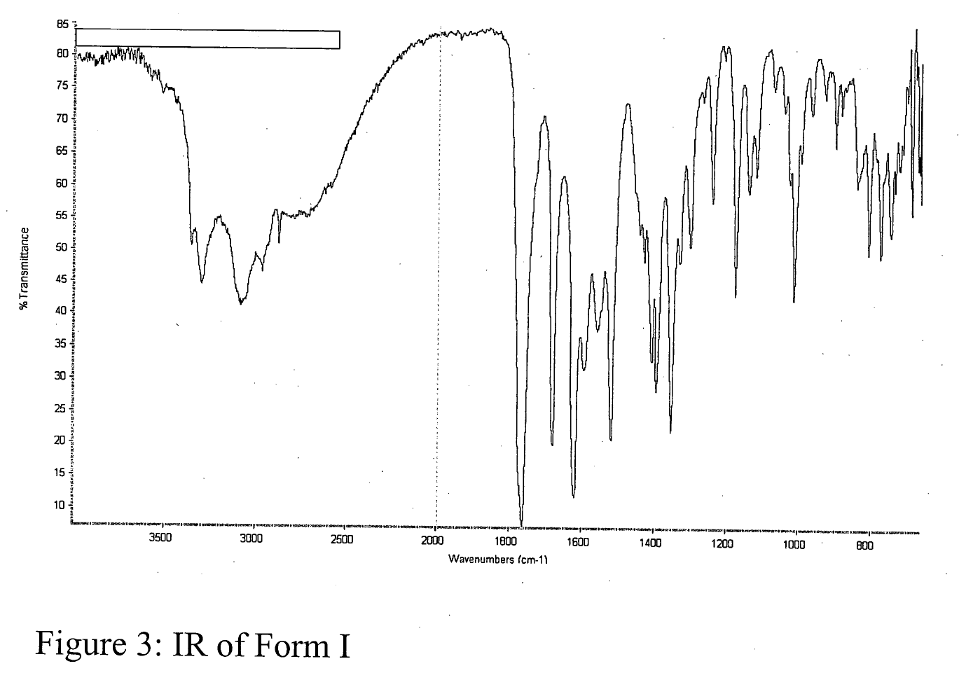

FIG. 4 is the infrared spectrum of the pyridine salt of Cefdinir.

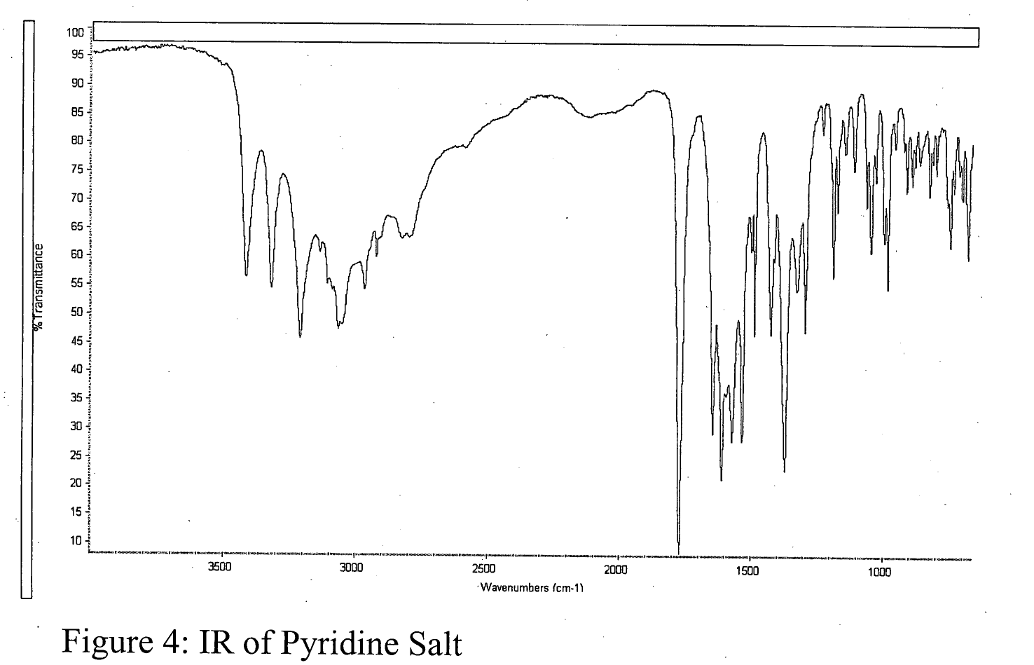

FIG. 5 is the TGA of the pyridine salt of Cefdinir.

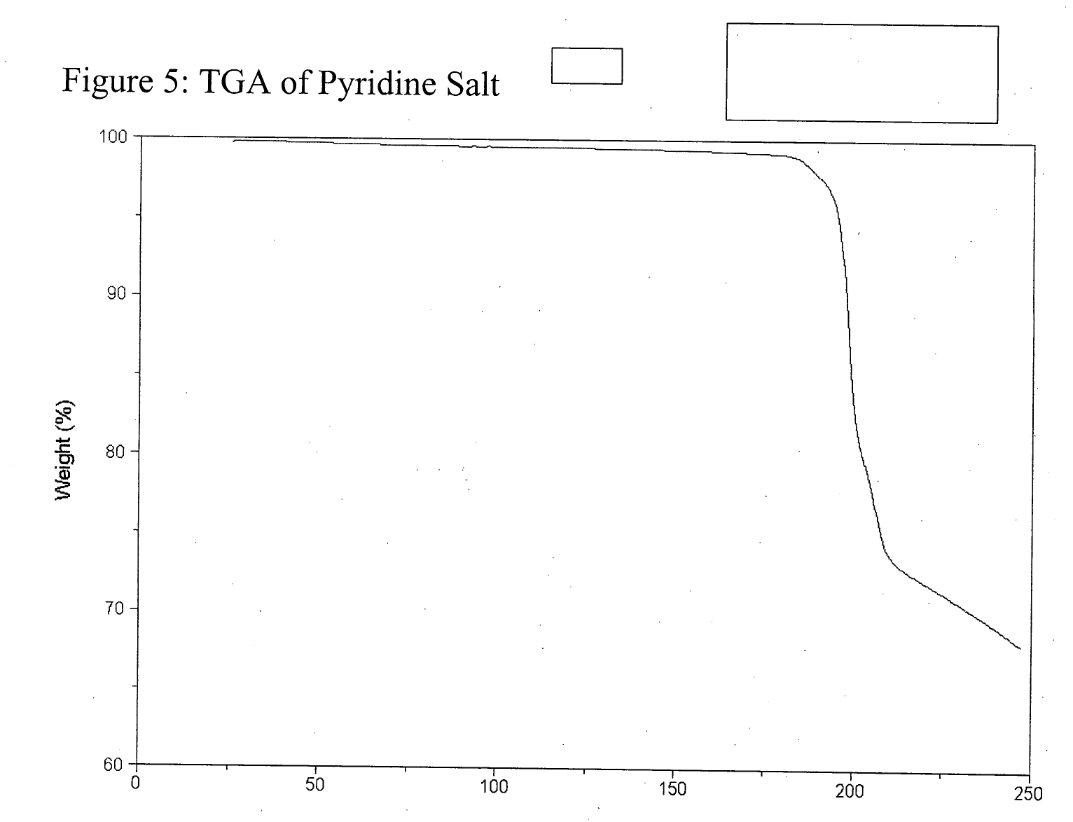

FIG. 6 is the ¹H NMR spectrum of the pyridine salt of Cefdinir.

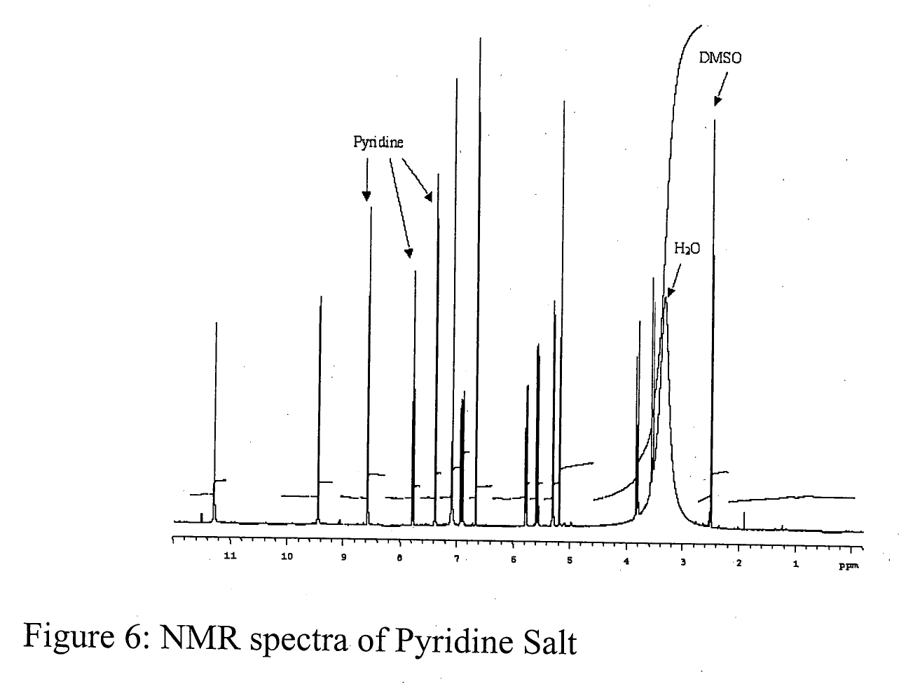

### SUMMARY OF THE INVENTION

The present invention describes a novel salt of Cefdinir. In its principle embodiment the present invention describes a pyridine salt of Cefdinir with characteristic peaks in the powder X-ray diffraction pattern at values of two theta of 8.1±0.1°, 10.7±0.1°, 12.1±0.1°, 13.7±0.1°, 17.8±0.1°, 19.0±0.1°, 20.4±0.1°, 21.5±0.1°, 22.2±0.1°, 23.0±0.1°, 24.3±0.1°, and 25.5±0.1°.

In another embodiment the present invention describes a pyridine salt of Cefdinir prepared by a process comprising suspending crystalline Form I of Cefdinir (preferably about 300 mg in excess of solubility) in pyridine for a period of time (preferably about 1 to about 8 weeks) followed by isolating the desired salt. This process may be conducted at about −5° C. to about 50° C. Preferably this process is conducted at about 20° C. to about 40° C., most preferably at about 23° C.

### DETAILED DESCRIPTION OF THE INVENTION

Powder X-ray diffraction was performed using an XDS-2000/X-ray diffractometer equipped with a 2 kW normal focus X-ray tube and a Peltier cooled germanium solid-state detector (Scintag Inc., Sunnyvale, Calif.). The data was processed using DMSNT software (version 1.37). The X-ray source was a copper filament operated at 45 kV and 40 mA. The alignment of the goniometer was checked daily using a Corundum standard. The sample was placed in a thin layer onto a zero background plate, and continuously scanned at a rate of 2° two-theta per minute over a range of 2 to 40° two-theta.

Characteristic powder X-ray diffraction pattern peak positions are reported in terms of the angular positions (two theta) with an allowable variability of ±0.1°. This allowable variability is specified by the U.S. Pharmacopeia, pages 1843-1884 (1995). The variability of ±0.1° is intended to be used when comparing two powder X-ray diffraction patterns. In practice, if a diffraction pattern peak from one pattern is assigned a range of angular positions (two theta) which is the measured peak position ±0.1° and if those ranges of peak positions overlap, then the two peaks are considered to have the same angular position (two theta). For example, if a diffraction pattern peak from one pattern is determined to have a peak position of 5.2°, for comparison purposes the allowable variability allows the peak to be assigned a position in the range of 5.1°-5.3°. If a comparison peak from the other diffraction pattern is determined to have a peak position of 5.3°, for comparison purposes the allowable variability allows the peak to be assigned a position in the range of 5.2°-5.4°. Because there is overlap between the two ranges of peak positions (i.e., 5.1°-5.3° and 5.2°-5.4°) the two peaks being compared are considered to have the same angular position (two theta).

Transmission infrared spectra of the solid was obtained using a Fourier-transform infrared spectrometer (Nicolet Magna 750 FT-IR Spectrometer, Nicolet Instrument Corporation, Madison, Wis.) equipped with a Nicolet NIC-PLAN microscope. The microscope had an MCT-A liquid nitrogen cooled detector. The sample was rolled on a 13 mm×1 mm BaF₂ disc sample holder; 64 scans were collected at 4 cm⁻¹ resolution.

Thermogravimetric analysis (TGA) was performed in TA Instruments TG2950 (TA Instruments, New Castle, Del.). The samples were scanned at 10° C./minute with a dry nitrogen purge at 60 mL/minute.

The ¹ H NMR spectrum of the pyridine salt of Cefdinir was recorded using a Unity 500 MHz spectorometer (Varia, Inc., Palo Alto, Calif.) operating at a proton frequency of 500.5 MHz and a sample temperature of 30° C. The spectrum was acquired using 64 transients, a 60° pulse, a specral width of 9497 Hz, 32768 data points, an acquisition time of 1.024 seconds, and a relaxion delay of 0.8 seconds. The sample was dissolved in 0.7 mL of DMSO-d₆.

In accordance with methods of treatment and pharmaceutical compositions of the invention, the compounds can be administered alone or in combination with other agents. When using the compounds, the specific therapeutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidently with the compound used. The compounds can be administered orally, parenterally, intranasally, rectally, vaginally, or topically in unit dosage formulations containing carriers, adjuvants, diluents, vehicles, or combinations thereof. The term “parenteral” includes infusion as well as subcutaneous, intravenous, intramuscular, and intrastemal injection.

Parenterally administered aqueous or oleaginous suspensions of the compounds can be formulated with dispersing, wetting, or suspending agents. The injectable preparation can also be an injectable solution or suspension in a diluent or solvent. Among the acceptable diluents or solvents employed are water, saline, Ringer's solution, buffers, monoglycerides, diglycerides, fatty acids such as oleic acid, and fixed oils such as monoglycerides or diglycerides.

The effect of parenterally administered compounds can be prolonged by slowing their release rates. One way to slow the release rate of a particular compound is administering injectable depot forms comprising suspensions of poorly soluble crystalline or otherwise water-insoluble forms of the compound. The release rate of the compound is dependent on its dissolution rate, which in turn, is dependent on its physical state. Another way to slow the release rate of a particular compound is administering injectable depot forms comprising the compound as an oleaginous solution or suspension. Yet another way to slow the release rate of a particular compound is administering injectable depot forms comprising microcapsule matrices of the compound trapped within liposomes, or biodegradable polymers such as polylactide-polyglycolide, polyorthoesters or polyanhydrides. Depending on the ratio of drug to polymer and the composition of the polymer, the rate of drug release can be controlled.

Transdermal patches can also provide controlled delivery of the compounds. The rate of release can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise excipients such as sucrose, lactose, starch, microcrystalline cellulose, mannitol, talc, silicon dioxide, polyvinylpyrrolidone, sodium starch glycolate, magnesium stearate, etc. Capsules, tablets and pills can also comprise buffering agents, and tablets and pills can be prepared with enteric coatings or other release-controlling coatings. Powders and sprays can also contain excipients such as talc, silicon dioxide, sucrose, lactose, starch, or mixtures thereof. Sprays can additionally contain customary propellants such as chlorofluorohydrocarbons or substitutes thereof.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents. Liquid dosage forms may also be contained within soft elastic capsules.

Topical dosage forms include ointments, pastes, creams, lotions, gels, powders, solutions, sprays, inhalants, and transdermal patches. The compound is mixed, if necessary under sterile conditions, with a carrier and any needed preservatives or buffers. These dosage forms can also include excipients such as animal and vegetable fats, oils, waxes, paraffins, starch, tragacanth, cellulose derivatives, polyethylene glycols, silicones, bentonites, talc and zinc oxide, or mixtures thereof. Suppositories for rectal or vaginal administration can be prepared by mixing the compounds with a suitable non-irritating excipient such as cocoa butter or polyethylene glycol, each of which is solid at ordinary temperature but fluid in the rectum or vagina. Ophthalmic formulations comprising eye drops, eye ointments, powders, and solutions are also contemplated as being within the scope of this invention.

The following example will serve to further illustrate the preparation of the pyridine salt of Cefdinir. Form I of Cefdinir was prepared according to the procedure described in U.S. Pat. No. 4,559,334 and U.S. Pat. No. 4,935,507, both of which are herein fully incorporated by reference.

Briefly, the process for the preparation of cefdinir is detailed below.

To a solution of benzhydryl 7-(4-bromoacetoacetamido)-3-vinyl-3-cephem-4-carboxylate (10 g) in a mixture of methylene chloride (70 ml) and acetic acid (25 ml) can be dropwise added isoamylnitrite (3.5 ml) at −3° to −5° C. The mixture can be stirred for 40 minutes at −5° C., followed by addition of acetylacetone (4 g) and stirring for 30 minutes at 5° C. To the reaction mixture can be added thiourea (3 g) and stirring for 3 hours, then added dropwise is ethyl acetate (70 ml) and diisopropyl ether (100 ml). The resultant precipitate can be collected by filtration and dried in vacuo to give benzhydryl 7-[2-(-aminothiazaol-4-yl)-2-hydroxyiminoacetamido]-3-vinyl-3-cephem-4-carboxylate hydrobromide (syn isomer) This product can be added portionwise to a mixture of 2,2,2-trifluroacetic acid and anisole at 5° to 7° C. After stirring for 1 hour at 5° C., the reaction mixture can be added dropwise to diisopropyl ether (150 ml). The resultant precipitate can be collected by filtration and dissolved in a mixture of terahydrofuran (10 ml) and ethyl acetate (10 ml). The organic layer can be extracted with an aqueous sodium bicarbonate. The aqueous extract washed with ethyl acetate while keeping the pH value at 5 and then adjusted to pH 2.2 with 10% hydrochloric acid. This solution can be stirred for 1 hour at 0° C., and the obtained crystals collected by filtration and dried in vacuo to give 7-[2-(2-aminothiazol-4-yl)-2-hydroxyiminoacetamido]-3-vinyl-3 cephem-4-carboxylic acid (syn isomer).

Alternatively, to a solution of benzhydryl 7-[2-(2-aminothiazol-4-yl)-2-hydroxyiminoacetamido]-3-vinyl-3-cephem-4-carboxylate (syn isomer) (5 g) in a mixture of anisole (20 ml) and acetic acid (5 ml) was added dropwise boron trufuloride etherate (5 ml) at 10° C. After stirring for 20 minutes at 10° C., the reaction mixture was poured into a mixture of tetrahydrofuran (100 ml), ethyl acetate (100 ml) and water (100 ml), and then adjusted to pH 6.0 with 20% aqueous sodium hydroxide. The resultant aqueous layer was separated and washed with ethyl acetate under keeping pH value at 6.0. This solution was subjected to chromatography on aluminum oxide.

The fractions eluted with 3% aqueous sodium acetate were collected and adjusted to pH 4.0 with 10% hydrochloric acid. This solution was further chromatographed on nonionic absorption resin “Diaion HP-20” (Trademark, manufactured by Mitsubishi Chemical Industries). The fractions eluted with 20% aqueous adjusted to pH 2.0 with 10% hydrochloric acid. The resultant precipitate was collected by filtration and dried in vacuo to give 7-[2-(2-aminotiazol-4-yl)-2-hydroxyminioacetamido]-3-vinyl-3-cephem-4-carboxylic acid (syn isomer).

Form I of Cefdinir

A pure cefdinir can be obtained by acidifying the solution containing cefdinir at room temperature or under warming and thereby having the crystals separate out of the solution.

Suitable examples of “the solution containing cefdinir may include, for example, an aqueous solution of the alkali metal salt of cefdinir. The solution containing cefdinir is acidified, if necessary, after said solution is subjected to a column chromatography on activated charcoal, nonionic adsorption resin, alumina, acidic aluminium oxide. The acidifying process can be carried out by adding an acid such as hydrochloric acid or the like preferably in the temperature range from room temperature to 40° C., more preferably, from 15° to 40° C. The amount of the acid to be added preferably makes the pH value of the solution from about 1 to about 4.

A pure cefdinir can be also obtained by dissolving the cefdinir in an alcohol (preferably methanol), continuing to stir this solution slowly under warming (preferably below 40° C.), preferably after the addition of water warmed at almost the same temperature as that of said solution, then cooling this solution to room temperature and allowing it to stand.

During the crystallization of cefdinir, it is preferable to keep the amount slightly beyond the saturation. Cefdinir obtained according to aforesaid process can be collected by filtration and dried by means of the conventional methods.

7-[2-(2-Aminothiazol-4-yl)-2-hydroxyminoacetamido]-3-vinyl-3-cephem-4-carboxylic acid (syn isomer) (29.55 g) can be added to water (300 ml) and the mixture adjusted to pH 6.0 with saturated sodium bicarbonate aqueous solution. The resultant solution can be subjected to a column chromatography on activated charcoal and eluted with 20% aqueous acetone. The fractions are combined and concentrated to a volume of 500 ml. The resultant solution pH is adjusted to 1.8 at 35° C. with 4N hydrochloric acid. The resultant precipitates are collected by filtration, washed with water and dried to give 7-[2-(2 aminothiazol-4-yl)-2-hydroxyminoacetamido]-3-vinyl-3-cephem-4-carboxylic acid (syn isomer).

Alternatively, to a solution of 7-[2-(2-aminothiazol-4-yl)-2-hydroxyminoacetamido]-3-vinyl-3-cephem-4-carboxylic acid (syn isomer) (0.5 g) in methanol (10 ml) can be added dropwise warm water (35° C.; 1.5 ml) at 35° C. and the resultant solution stirred slowly for 3 minutes, then allowed to stand at room temperature. The resultant crystals are collected by filtration, washed with water and then dried to give 7-[2(2-3-aminothiazol-4-yl)-2-hydroxyminioacetamido]3-vinyl-3-cephem-4-carboxylic acid (syn isomer) as crystals.

Preparation of Novel Cefdinir Polymorph from Pyridine

The solubility of Cefdinir Form I in pyridine was estimated. A suspension of Cefdinir Form I (total of approx. 500 mg or 300 mg in excess of the solubility) in 4 mL of pyridine was allowed to stand at room temperature. After 1 week, the solid from the suspension was separated and the powder X-ray diffraction pattern, ¹H NMR, TGA, and infrared spectrum of the moist solid were generated.

The foregoing is merely illustrative of the invention and is not intended to limit the invention to the disclosed embodiments. Variations and changes which are obvious to one skilled in the art are intended to be within the scope and nature of the invention which are defined in the appended claims.

1. A pyridine salt of Cefdinir with characteristic peaks in the powder X-ray diffraction pattern at values of two theta of 8.1±0.1°, 10.7±0.1°, 12.1±0.1°, 13.7±0.1°, 17.8±0.1°, 19.0±0.1°, 20.4±0.1°, 21.5±0.1°, 22.2±0.1°, 23.0±0.1°, 24.3±0.1°, and 25.5±0.1°.

2. A pyridine salt of Cefdinir prepared by a process comprising:
(a) suspending Form I of Cefdinir in pyridine; and
(b) isolating the desired salt from the suspension of step (a).

3. The salt of claim 2 wherein the suspension of step (a) has about 300 mg of Form I of Cefdinir.

4. The salt of claim 2 wherein step (a) is conducted at about −5° C. to about 50° C.

5. The salt of claim 2 wherein step (a) is conducted at about 20 to about 40° C.

6. The salt of claim 2 wherein step (a) is conducted at about 23° C.

7. A process for preparing a pyridine salt of Cefdinir, the process comprising:
(a) suspending Form I of Cefdinir in pyridine;
(b) isolating the desired polymorph from the suspension of step (a).

8. The process of claim 7 wherein the suspension of step (a) has about 300 mg of Form I of Cefdinir.

9. The process of claim 7 wherein step (a) is conducted at about 20° C. to about 40° C.

10. The process of claim 7 wherein step (a) is conducted at about 23° C.

11. The process of claim 7 wherein step (a) is conducted for about 1 to about 8 weeks.

12. A pharmaceutical composition comprising the salt of claim 1 in combination with a pharmaceutically acceptable carrier.
